(19) United States
(12) Patent Application Publication
(10) Pub. No.: US 2009/0171266 A1
(43) Pub. Date: Jul. 2, 2009

(54) COMBINATION THERAPY

(76) Inventor: Dagan Harris, Hadera (IL)

Correspondence Address: EMPK & Shiloh, LLP;c/o Landon IP, Inc., 1700 Diagonal Road, Suite 450, Alexandria, VA, 22314, US

(21) Appl. No.: 11/968,169

(22) Filed: Jan. 1, 2008

(51) Int. Cl. A61B 8/00; A61M 5/00

(52) U.S. Cl. 604/22

(57) ABSTRACT

There is provided a method for producing lysis of adipose tissue that includes applying focused ultrasonic energy to the adipose tissue and applying at least one additional therapeutic procedure to said adipose tissue or surrounding tissue. There is further provided a system for combination therapy for producing lysis of adipose tissue that includes a transducer adapted to apply focused ultrasonic energy to said adipose tissue and at least one additional unit adapted to provide a therapeutic procedure to said adipose tissue or surrounding tissue(s).

### BACKGROUND

The esthetic medical field is a fast growing area in which medical procedures as well as medical devices are used to promote aesthetic traits. One of the most popular areas in the aesthetic medical field is the removal and/or reduction of the number of subcutaneous fat cells and the volume of adipose tissue.

Various methods, techniques and procedures have been described in the art, for their use in removal and/or reducing the number and/or volume of subcutaneous fat cells and adipose tissue. The methods may include invasive surgical procedures, such as liposuction, as well as non invasive procedures, such as, for example, the use of medications, ointments, laser based procedures, radio-frequency based procedures, infra-red based procedures, ultra sound based procedures and the like.

### SUMMARY

The following embodiments and aspects thereof are described and illustrated in conjunction with systems, tools and methods which are meant to be exemplary and illustrative, not limiting in scope. In various embodiments, one or more of the above-described problems have been reduced or eliminated, while other embodiments are directed to other advantages or improvements.

According to some embodiments, there is provided a method for producing lysis of adipose tissue comprising combination therapy that includes: applying focused ultrasonic energy to said adipose tissue and applying at least one additional therapeutic procedure to said adipose tissue or surrounding tissue.

According to some embodiments, there is provided a system for combination therapy for producing lysis of adipose tissue that includes a transducer adapted to apply focused ultrasonic energy to said adipose tissue and at least one additional unit adapted to provide a therapeutic procedure to said adipose tissue or surrounding tissue(s).

According to further embodiments, there is provided a method for producing lysis of adipose tissue comprising combination therapy that includes: applying surface ultrasonic energy to said adipose tissue and applying at least one additional therapeutic procedure to said adipose tissue or surrounding tissue.

According to some embodiments, there is provided a system for combination therapy for producing lysis of adipose tissue that includes a transducer adapted to apply surface ultrasonic energy to said adipose tissue and at least one additional unit adapted to provide a therapeutic procedure to said adipose tissue or surrounding tissue(s).

According to some embodiments, at least one additional therapeutic procedure in the method for producing lysis of adipose tissue may include: administration of a substance, invasive surgical procedure, non-invasive procedure and any combination thereof.

According to some embodiments, the substance administered may include a drug, toxin, enzyme, biomolecule, inert fluid, molecule and any combination thereof and may be administered by any administration route, such as injection, ingestion, inhalation, topical application and any combination thereof. The substance may be in the form of, for example, but not limited to, fluids, liquids, gels, ointments, creams, pharmaceutical compositions, microparticles and any combination thereof. The substance may include, for example: botulinum toxin type A, collagenase, adiponectin, saline, gas bubbles, alpha-2 adrenergic inhibitor and any combination thereof.

According to some embodiments, the invasive surgical procedure may include liposuction, ultrasound assisted liposuction, external ultrasonic assisted liposuction, cryosurgery and any combination thereof.

According to some embodiments, the non invasive procedure may include laser-based procedures, radio frequency based procedures, Infra Red based procedures, microwave based procedures, massaging based procedures and any combination thereof.

According to some embodiments, the focused ultrasonic energy may be applied before the at least one additional therapeutic procedure is applied. The focused ultrasonic energy is applied at the same time as the at least one additional therapeutic procedure is applied. The focused ultrasonic energy is applied after the at least one additional therapeutic procedure is applied.

According to some embodiments, the surface ultrasonic energy may be applied before the at least one additional therapeutic procedure is applied. The surface ultrasonic energy is applied at the same time as the at least one additional therapeutic procedure is applied. The surface ultrasonic energy is applied after the at least one additional therapeutic procedure is applied.

According to some embodiments, the method for producing lysis of adipose tissue may result in an enhanced effect on said adipose tissue. The enhanced effect may be determined/measured by various parameters, such as temporal parameters, biological parameters, physiological parameters, spatial parameters and any combination thereof. The enhanced effect may be determined by comparing the effect achieved by the combination therapy to the effect achieved by each of the procedures of the combination therapy when the procedures are performed alone and not in combination with other procedures.

According to some embodiments, the enhanced effect may include increasing the lysis of said adipose tissue. According to some embodiments, the enhanced effect may include decreasing treatment time needed to produce adipose tissue lysis.

According to some embodiments, the enhanced effect may be a synergistic effect. The enhanced effect may be an additive effect.

According to some embodiments, the method may further effect surrounding tissue such as muscle tissue, connective tissue, blood vessels, nerve tissue, fat tissue, adipose tissue and any combination thereof.

In addition to the exemplary aspects and embodiments described above, further aspects and embodiments will become apparent by reference to the figures and by study of the following detailed descriptions.

### BRIEF DESCRIPTION OF THE FIGURES

Examples illustrative of embodiments are described below with reference to figures attached hereto. In the figures, identical structures, elements or parts that appear in more than one figure are generally labeled with a same numeral in all the figures in which they appear. Dimensions of components and features shown in the figures are generally chosen for convenience and clarity of presentation and are not necessarily shown to scale. The figures are listed below.

FIGS. 1A-L—illustrations of exemplary flow diagrams of a method of combination therapy, according to some embodiments;

FIGS. 2A-E—illustrations of exemplary flow diagrams of a method of combination therapy, according to some embodiments; and

FIG. 3—a schematic illustration of a system for combination therapy, according to some embodiments

### DETAILED DESCRIPTION

In the following description, various aspects of the disclosure will be described. For the purpose of explanation, specific configurations and details are set forth in order to provide a thorough understanding of the disclosure. However, it will also be apparent to one skilled in the art that the disclosure may be practiced without specific details being presented herein. Furthermore, well-known features may be omitted or simplified in order not to obscure the embodiments.

As referred to herein, the terms “therapeutic procedure”, “therapeutic treatment”, “therapeutic method”, “therapeutic substance”, “procedure”, “therapy”, “therapeutic material”, “treatment” may interchangeably be used and may include any method, procedure, treatment, substance, material and the like that may be used to affect cells and tissue.

As referred to herein, the term “affect” in the context of cells and tissue may include any effect (beneficial, deleterious and/or harmless) on cells and tissue. According to some embodiments, the effect may be a deleterious effect and may include at least a partial damage to cells and/or tissue. Deleterious effect and/or damage to cells and tissue may include for example, but is not limited to: destruction, lysis, death (apoptosis and necrosis), cavitation, removal, reduction in number, reduction in size, reduction in distribution, reduction in cell mass, reduction in cell content, reduction in fat percentage, change in structure, change in composition and any combination thereof.

As referred to herein, the term “enhanced effect” in the context of combination therapy may include an effect whose results are enhanced (become more advantageous) as compared to the effect of each therapy when it is performed individually (alone and not in combination with other therapies). Enhancement may include any improvement of the effect of the various therapies that may result in an advantageous result as compared to the results achieved by the therapies when performed alone (not in combination with any other therapy). Enhanced effect and determination of enhanced effect may be measured by various parameters that may include: temporal parameters, such as, for example, time, length of treatment, recovery time, long-term (chronic) effect of the treatment, reversibility of treatment; biological parameters such as, for example, cell number, cell volume, cell composition, tissue volume, tissue size, tissue composition and the like; spatial parameters that may include, for example, tissue strength, tissue size, tissue accessibility, and the like; physiological parameters such as, for example, body contouring, pain, discomfort, recovery time, visible marks (such as scars) and the like. Enhanced effect may include a synergistic enhancement, that may include an enhanced effect whose result is more than the additive effects of each therapy by itself. Enhanced effect may include an additive enhancement that may include an enhanced effect whose result is substantially equal to the additive effect of each therapy when performed by itself. Enhanced effect may include less than synergistic effect, wherein the enhanced effect is lower than the additive effect of each therapy when performed by itself, but still better than the effect of each therapy when performed by itself.

As referred to herein the terms “thereat” and “simultaneously” may interchangeably be used and may include any time frame of between about 0 seconds to 10 minutes.

As referred to herein, the terms “given” and “performed” in the context of therapeutic procedure(s) may interchangeably be used and may mean the time at which the treatment(s) was/were applied (started, commenced).

As refereed to herein the terms “therapeutic” and “aesthetic”, “cosmetic”, “medical” procedures may interchangeably be used and may relate to any type of procedure, treatment, and the like that are described below herein.

As referred to herein, the terms “target area”, target volume” and “target tissue” may interchangeably be used.

As referred to herein, the terms “fat cells”, “fat tissue”, “fat deposits”, “adipose tissue” “adipocytes” and “cellulite” may interchangeably be used.

As referred to herein, the term Focused Ultrasound (FU) is related to ultrasonic energy that may be externally and non-invasively applied to a surface in a focused manner such that the energy is focused to a specified internal target area. The ultrasonic (acoustic) energy applied may be, for example, in the form of waves. Applying and focusing the ultrasonic energy may be performed by various ways, such as, for example, by an ultrasonic transducer that may be adapted to focus the ultrasonic energy. The focused ultrasonic energy may include various intensity levels, such as in the range of 100 to 1500 kHz. For example, FU may include High Intensity Focused Ultrasound (known in the art as HIFU). For example, the FU may be applied to/on a subject skin and focused on a subcutaneous target area/volume, such as fat cells and adipose tissue. The terms “Focused Ultrasound”, “FU”, “Focused Ultrasound treatment” and “FU treatment” may interchangeably be used herein.

As referred to herein, the term Surface Ultrasound (SU) relates to ultrasonic (acoustic) energy that may be externally and non-invasively applied at, or in close proximity to a surface and aimed towards an internal target area and/or target volume in a non-focused manner. The ultrasonic energy may be, for example, in the form of waves. Applying and focusing the ultrasonic energy may be performed by various ways, such as for example by an ultrasonic transducer. The surface ultrasound energy may include various intensity levels, such as in the range of 100 to 1500 kHz. The SU may be applied externally at an external surface, to target an internal area/volume. For example, the SU may be applied to a subject skin to target subcutaneous target area/volume. The terms “Surfaced Ultrasound”, “SU”, “Surface Ultrasound treatment” and “SU treatment” may interchangeably be used herein.

The interaction of ultrasonic energy (such as FU and SU) with a target area, (such as, for example, tissue), may result in heating and/or cavitation that may lead to at least a partial damage to the target area. When the ultrasonic energy is absorbed by, for example, tissue, it may be converted to heat. Such heating of the tissue may result in at least partial damage to the tissue. Heating as a result of ultrasonic energy is not tissue specific and all tissue within the target area/volume may be affected. The term cavitation is generally used to describe the behavior of voids or bubbles in a liquid. Cavitation is usually divided into two classes: inertial (transient), which is the process where a void or bubble in a liquid rapidly collapses, producing a shock wave; and non-inertial cavitation, which is a process where a bubble in a fluid is forced to oscillate in size or shape due to energy input, such as an acoustic field. In the context of a biological environment, the liquid in which the cavitation phenomena may occur may include, for example, cells, tissue and the like. Thus, the cavitation phenomena caused by, for example, the absorption of ultrasonic energy by tissue may result in damage to the tissue and may be tissue specific, since the cavitation process may depend on specific tissue characteristics.

Ultrasonic energy such as FU and SU may be used to affect subcutaneous fat cells and tissue. The ultrasonic energy that may externally and non-invasively be applied, may be absorbed by the subcutaneous fat tissue. As a result of absorption of the ultrasonic energy by the subcutaneous fat tissue, a deleterious effect may be achieved and, at least partial damage to the fat tissue may be obtained.

Combination Therapy with FU Treatment

According to some embodiments, there is provided a combination therapy that may include FU treatment and at least one additional therapeutic procedure (treatment) that may be used in combination with the FU treatment. The combination therapy may be used to affect fat cells and tissue by providing a deleterious effect to the fat cells and tissue. The FU treatment and the at least one additional treatment may be given at the exact same location (region), at locations (regions) that are in close proximity, at regions that are distant (such as different body parts), and any combination thereof. The deleterious effect on fat cells and tissue that may be achieved by the combination therapy may be an enhanced effect. Enhanced effect and determination of enhanced effect may be measured by various parameters that may include, for example: temporal parameters, such as time, length of treatment, recovery time, long-term (chronic) effect of the treatment, reversibility of the treatment, and the like. For example, enhanced effect as determined by time parameter may be determined, for example, if the time needed to achieve a desired effect is shorter in the combination therapy as compared to the time the effect may be achieved by each therapy when performed individually (alone and not in combination); biological parameters such as cell number, cell volume, tissue volume, tissue size and the like; spatial parameters that may include, for example, tissue strength, tissue size, tissue accessibility, and the like; physiological parameters such as body contouring, pain, discomfort, recovery time, visible marks (such as scars) and the like. Enhanced effect may be determined by comparing the effect achieved by the combination therapy to the effect that may be achieved by each of the procedures when the procedures are performed individually (alone, not in combination). An enhanced effect may include a synergistic effect, wherein the effect achieved by the combination therapy is greater than the additive sum of the effects achieved by each of the procedures when performed individually. An enhanced effect may include an additive effect, wherein the effect achieved by the combination therapy is substantially equal to the additive sum of the effects achieved by each of the procedures when performed individually. An enhanced effect may include a less than additive effect, wherein the effect achieved by the combination therapy is less than the additive sum of the effects achieved by each of the procedures when performed individually, but is still greater than the effect achieved by each of the procedures when performed individually.

According to some embodiments, the combination therapy may be performed at any time period in the range of about 4 months before the FU treatment to about 4 months after the FU treatment. For example, the combination therapy may be performed simultaneously, wherein the FU treatment and the at least one additional therapeutic procedure (treatment) are performed substantially at the same time, within the range of about 0 to 60 seconds to 10 minutes. The combination therapy may be performed such that the FU treatment is performed before the at least one additional therapeutic procedure is performed. The therapeutic procedure may be performed at a time difference in the range of between about 10 minutes to about four months after the FU treatment was performed. The combination therapy may be performed such that the FU treatment is performed after the at least one additional therapeutic procedure was given. The therapeutic procedure may be performed at a time difference in the range of between about 10 minutes to about four months before the FU treatment is performed.

According to some embodiments, the combination therapy may include FU treatment and administration of at least one substance/material. The substance/material that may be administered may be in the form of, for example, but is not limited to: fluids, liquids, gels, ointments, creams, pharmaceutical compositions, microparticles and any combination thereof. Administration may include any administration route, such as, for example, but is not limited to: ingestion, inhalation, injection, subcutaneous injection, anointment, topical administration, spreading and any combination thereof.

According to some embodiments, administration of the substance/material may be performed at the same time as the FU is performed. Administration of the substance material may be performed before or after the FU is performed.

According to some embodiments, the substance that may be administered in combination with FU treatment may include an active substance, such as a drug, toxin, enzyme, biomolecule, molecule, and the like and any combination thereof. The active substance may be in the form of pharmaceutical composition and may include an active substance that may be directed against fat cells, surrounding tissue, such as connective tissue, muscle tissue, nerves, blood vessels and the like, and any combination thereof. For example, the active substance that may be used in combination therapy may include, for example, botulinum toxin type A. Botulinum toxin A is widely used in cosmetics and esthetic medicine, usually in a form known as Botox. One of the effects of Botulinum toxin A is upon muscles, wherein the toxin has the ability to decrease muscle activity by blocking the release of acetylcholine at the neuromuscular junction, thereby rendering the muscle unable to contract for a period of time. In accordance, minute amounts of botulinum toxin A may be administered by injection (hereinafter, botulinum treatment) into the muscle tissue that is located in close proximity to the adipose tissue that may be targeted by the FU. Localized administration of botulinum toxin A (such as in the form of Botox) may include any number of units of Botox, such as in the range of about 1 to 600 units. For example, 20 to 400 units of Botox may be administered. For example, about 50 to 300 units of Botox may be administered. For example, about 100 to 200 units of Botox may be administered. The Botox may be administered at various time frames in relation to the FU treatment. For example, the Botox may be administered prior to the FU treatment. For example, the Botox may be administered up to six weeks prior to the FU treatment. For example, the Botox may be administered four weeks prior to the FU treatment. For example, the Botox may be administered two weeks prior to the FU treatment. The Botox may be administered thereat with the FU treatment. For example, the Botox may be administered on the same day as the FU treatment, in the range of about 6 hours before the FU treatment to about 6 hours after the FU treatment. The Botox may be administered at any time frame after the FU treatment. For example, the Botox may be administered up to about 6 weeks after the FU treatment. For example, the Botox may be administered about four weeks after the FU treatment. For example, the Botox may be administered about 2 weeks after the FU treatment. For example, the Botox may be administered about 6 days after the FU treatment. As a result of the botulinum treatment, the muscles surrounding the adipose tissue may be relaxed, making the adipose tissue more accessible to the FU treatment. The effect of the combination therapy may result in a higher number of fat cell deaths and in a larger volume of fat tissue that has been lysed. In addition, the length of treatment needed to obtain a desirable effect may be shorter in the combination therapy than that needed to obtain a similar effect by each treatment by itself. In addition, the long-term (chronic) effect of the treatment may be enhanced.

According to some embodiments, the substance that may be administered in combination with FU treatment may include an active substance. The active substance that may be used in combination therapy may include, for example, an enzyme, such as Collagenase. Collagenase is a well-known enzyme that may break (proteolyse) the protein collagen, which is the major constituent of connecting tissue. In accordance, Collagenase may be administered by injection (hereinafter, Collagenase treatment) into the connecting tissue that is located in close proximity to the adipose tissue that may be targeted by the FU. Localized administration of Collagenase may include 0.05 to 3.0 mg (milligram) per injection. For example, 0.1 to 2 mg of Collagenase may be administered. For example, 0.3 to 1.5 mg of Collagenase may be administered. For example, 0.5 to 1 mg of Collagenase may be administered. The Collagenase may be administered at any time frame relative to the FU treatment. For example, the Collagenase may be administered up to 14 weeks before the FU treatment. For example, the Collagenase may be administered about 12 weeks before the FU treatment. For example, the Collagenase may be administered about 10 weeks before the FU treatment. For example, the Collagenase may be administered about 8 weeks before the FU treatment. For example, the Collagenase may be administered about 6 weeks before the FU treatment. For example, the Collagenase may be administered about 4 weeks before the FU treatment. For example, the Collagenase may be administered about 2 weeks before the FU treatment. The Collagenase may be administered thereat with the FU treatment. For example, the Collagenase may be administered on the same day as the FU treatment, in the range of about 6 hours before the FU treatment to about 6 hours after the FU treatment. The Collagenase may be administered at any time frame after the FU treatment. For example, the Collagenase may be administered up to about 14 weeks after the FU treatment. For example, the Collagenase may be administered about 12 weeks after the FU treatment. For example, the Collagenase may be administered about 10 weeks after the FU treatment. For example, the Collagenase may be administered about 8 weeks after the FU treatment. For example, the Collagenase may be administered about 6 weeks after the FU treatment. For example, the Collagenase may be administered about 4 weeks after the FU treatment. For example, the Collagenase may be administered about 2 weeks after the FU treatment. For example, the Collagenase may be administered about 7 days after the FU treatment. For example, the Collagenase may be administered up to about 6 days after the FU treatment. As a result of the Collagenase treatment, the connective tissue surrounding the adipose tissue are at least partially relaxed, making the adipose tissue more accessible to the FU treatment. In another mode of operation, Collagenase may be administered by injection (hereinafter, Collagenase treatment) into the fat tissue that is targeted by the FU. As a result of the Collagenase treatment, the fat tissue may be more accessible to the FU treatment. The effect of the combination therapy may result in a higher number of fat cell deaths and in a larger volume of fat tissue that has been lysed. In addition, the length of treatment needed to obtain a desirable effect may be shorter in the combination therapy than that needed to obtain a similar effect by each treatment by itself. In addition, the long-term (chronic) effect of the treatment may be enhanced.

According to some embodiments, the active substance that may be used in combination therapy may include, for example, a protein, such as Adiponectin. Adiponectin is a protein that may modulate fat cells and has been suggested as having the ability to decrease fat in adipocytes or the number of adipocytes (such as described in PCT publication No. WO 02/072149). In accordance, Adiponectin may be administered by, for example, injection, enteral and/or parenteral routes (hereinafter, Adiponectin treatment) into the adipose tissue that is be targeted by the FU. Adiponectin may be administered at any time period relative to the FU treatment. For example, Adiponectin may be administered about 6 weeks before the FU treatment to about 6 weeks after the FU treatment. The effect of the combination therapy may result in a higher number of fat cell deaths and in a larger volume of fat tissue that has been lysed. In addition, the length of treatment needed to obtain a desirable effect may be shorter in the combination therapy than that needed to obtain a similar effect by each treatment by itself. In addition, the long-term (chronic) effect of the treatment may be enhanced.

According to some embodiments, the substance that may be administered in combination therapy with FU may include an inert fluid, such as a saline. Saline may be administered by injection into the fat tissue that is to be targeted by FU. Saline may be injected into the fat tissue at any tonicity range, such as, for example, isotonic, hypertonic and/or hypotonic. Saline injection into the tissue may result in spatial separation of surrounding tissue from fat tissue, malting the fat tissue more accessible to FU treatment, by creating a more advantageous surface. Moreover, due to the spatial separation of fat tissue from surrounding tissue, better propagation of the ultrasound energy (waves) may be achieved. As a result, an enhanced effect may be observed. The effect of the combination therapy may result in a higher number of fat cell deaths and in a larger volume of fat tissue that has been lysed. In addition, the length of treatment needed to obtain a desirable effect may be shorter in the combination therapy than that needed to obtain a similar effect by each treatment by itself. In addition, the long-term (chronic) effect of the treatment may be enhanced.

According to some embodiments, the substance that may be used in combination therapy with FU may include, for example, fluid at its gaseous state of aggregation, such as, for example, an inert gas, gas bubbles, a precursor of gas bubbles (such as described in U.S. Pat. No. 5,219,401), and the like. The fluid at its gaseous state of aggregation may be administered by, for example, injection, to the fat tissue and upon FU treatment that may be given immediately; the gas bubbles may explode (as a result of the acoustic energy received by the tissue), and thus the effect of FU treatment may be augmented. For example, the precursor of gas bubbles may be injected into the fat tissue, and may be converted to gas bubbles. The tissue may then be exposed to FU treatment and as a result of the acoustic energy received by the tissue, the gas bubbles may implode. The FU treatment may be given immediately after the fluid had been injected, such as, for example in the range of 1 second to 1 hour. As a result of the combination therapy, damage to the fat tissue may be achieved. The damage to the tissue may be observed as a number of fat cells that have been damaged and/or reduction in fat tissue volume. Alternatively, the combination therapy may also be enhanced by temporal parameters, that is, the treatment time with FU needed to get a desired damage (such as number of fat cells that have been damaged and/or reduction in fat tissue volume, and the like.) is shorter in the combination therapy, than the time needed to achieve the same effect when each therapy is performed alone. In addition, the long-term (chronic) effect of the treatment may be enhanced.

According to some embodiments, the substance that may be administered in combination therapy with FU may include ointments that may be used for the reduction of fat deposits. The ointment may include active substances (ingredients) that may be used for selective reduction of regional fat deposits. The active ingredients may include, for example, but are not limited to: Caffeine, L-camitine, Algeae Extracts, Bladderwrack extract, Capsium extract, Green tea extract, Retinol A, Shea Butter, Horsetail Extract, Caprylic triglyceride, Cyclomethicon, Dimethicone, Hydrogenated lecitin, Acrylates C10-30 Alkyl Acrylate crosspolymer, Imidazolidinyl Urea, Triethanolamine, Methylparaben, and the like, and any combinations thereof. For example, the ointment may include commercially available anticellulite creams, such as, for example, Revitol, Sculpt and Tone™, and the like that may include various combinations of active substances that may be used for selective reduction of regional fat deposits. For example, the ointment may further include alpha-2 adrenergic inhibitor (such as described in U.S. Pat. No. 4,588,724). In accordance, the ointment may be administered by, for example, topical spreading (hereinafter ointment treatment) on the skin region, which resides in correlation to the location of the fat tissue that is to be targeted by the FU treatments. Localized administration of ointment may include topical administration of the ointment, such as by massaging the ointment for 30 to 60 seconds, until at least partially absorbed. The ointment may be administered at any time frame relative to the FU treatment, such as any time in the range of about 60 days before the FU treatment to about 60 days after the FU treatment. The effect of the combination therapy may result in a higher number of fat cell deaths and in a larger volume of fat tissue that has been lysed. In addition, the length of treatment needed to obtain a desirable effect may be shorter in the combination therapy than that needed to obtain a similar effect by each treatment by itself. In addition, the long-term (chronic) effect of the treatment may be enhanced.

According to some embodiments, the combination therapy may include FU treatment in addition to at least one surgical/invasive procedure. The invasive procedure may include, for example, but is not limited to: procedures such as liposuction, ultrasound assisted liposuction (UAL), external ultrasound assisted liposuction (EUAL), cryosurgery, and the like.

According to some embodiments, the combination therapy may include FU treatment along with liposuction procedure. Liposuction is a medical procedure that involves surgical removal of all or part of the subcutaneous fat cell layer in target areas. This invasive procedure involves local or general anesthesia and engages the insertion of, for example, a cannule through a small skin incision into the adipose tissue and thus the fat may then be suctioned out. The cannule may be moved back and forth in different tissue levels covering the volume to be suctioned. The fat is torn and evacuated at the same time. FU may be performed at any time frame before the liposuction procedure. In this mode, fat cells and tissue that are damaged by the FU treatment may be evacuated as part of the liposuction procedure. The effect of the combination therapy may result in a higher number of fat cell deaths and in a larger volume of fat tissue that has been lysed. In addition, the length of treatment needed to obtain a desirable effect may be shorter in the combination therapy than that needed to obtain a similar effect by each treatment by itself. In addition, the long-term (chronic) effect of the treatment may be enhanced.

According to some embodiments, the combination therapy may include FU treatment along with ultrasound assisted lipoplasty (UAL) procedure. UAL, which is related to liposuction may engage the use of a cannule that has an ultrasound probe at its tip; when energy is applied, the tissue next to the tip may be destroyed. The fat that is destroyed by the procedure may be evacuated by the same, or another cannule. FU treatment may be performed at any time frame relative to the UAL procedure. For example, the FU treatment may be performed up to about 8 weeks before the UAL procedure. In this mode, fat cells and tissue that are damaged by the FU treatment may be evacuated as part of the UAL procedure, which may be performed, for example, at a frequency of about 20-50 kHz with a power of 10-300 W/cm². The effect of the combination therapy may result in a higher number of fat cell deaths and in a larger volume of fat tissue that has been lysed. In addition, the length of treatment needed to obtain a desirable effect may be shorter in the combination therapy than that needed to obtain a similar effect by each treatment by itself. In addition, the long-term (chronic) effect of the treatment may be enhanced.

According to some embodiments, the combination therapy may include FU treatment with cryosurgery procedure. Cryosurgery is a procedure for destroying tissue, by freezing the tissue to be destroyed. This is a minimally invasive technique wherein a cryogen cooled probe may be inserted into a desired tissue, consequently freezing the tissue. For example, U.S. Pat. No. 6,032,675 discloses a method for removing fatty tissue in a body, combining cryosurgery and liposuction. In accordance, FU treatment may be performed at any time period relative to the cryosurgery procedure. For example, the FU treatment may be performed at the time range of about four months before the cryosurgery procedure to about four months after the cryosurgery procedure. In this mode, fat cells and tissue are damaged by the FU treatment and the cryosurgery procedure. Thus, the effect of the combination therapy may result in a higher number of fat cell deaths and in a larger volume of fat tissue that has been lysed. In addition, the length of treatment needed to obtain a desirable effect may be shorter in the combination therapy than that needed to obtain a similar effect by each treatment by itself. In addition, the long-term (chronic) effect of the treatment may be enhanced.

According to some embodiments, the combination therapy may include FU treatment in addition to at least one non-invasive procedure. The non-invasive procedure may include, for example, energy based procedures such as laser based procedure, infra-red based procedure, microwave-energy based procedure, radio frequency based procedure, and the like. The non-invasive procedure may include, for example, physical contact, direct contact, and the like, that may be applied to an outer surface, such as skin, that corresponds to the subcutaneous fat tissue that may be targeted by the FU treatment.

According to some embodiments, the combination therapy may include FU treatment performed along with a laser-based procedure. Laser based procedure may include, for example, the external application of laser energy thorough the skin to target areas in the body, such as fat tissue. For example, US patent application No. 2005/0203594 discloses a non invasive method of reducing fat from targeted regions of a patient's body by applying low-level laser energy externally through the skin of the patient to the targeted areas. In accordance, FU treatment may be performed at any time frame relative to the application of the laser energy (hereinafter, laser procedure). For example, the laser procedure may be performed in the range of about 8 weeks before the FU treatment to about 8 weeks after the FU treatment. The laser procedure may be performed at various wavelengths, such as, for example, in the range of about 515 to 1200 nm. For example, the laser procedure may be performed with a wavelength in the range of about 550 to 900 nm. For example, the laser procedure may be performed with a wavelength in the range of about 600 to 800 nm. For example, the laser procedure may be performed with a wavelength of about 635 nm. The laser procedure may be performed with a pulse duration range of about 0.2 to 100 milliseconds. For example, the laser procedure may be performed with a pulse duration range of about 0.5 to 88.5 milliseconds. For example, the laser procedure may be performed with a pulse duration range of about 1 to 75 milliseconds. For example, the laser procedure may be performed with a pulse duration range of about 20 to 50 milliseconds. For example, the laser procedure may be performed with a pulse duration range of about 30 to 40 milliseconds. The laser procedure may be performed with a pulse duration range of about 1 to 100 mJ/cm². For example, the laser procedure may be performed with a pulse duration range of about 3 to 70 mJ/cm². For example, the laser procedure may be performed with a pulse duration range of about 20 to 50 mJ/cm². For example, the laser procedure may be performed with a pulse duration range of about 30 to 40 mJ/cm². The FU treatment and the laser procedure may be directed to the same or different target area. The effect of the combination therapy may result in a higher number of fat cell deaths and in a larger volume of fat tissue that has been lysed. In addition, the length of treatment needed to obtain a desirable effect may be shorter in the combination therapy than that needed to obtain a similar effect by each treatment by itself. In addition, the long-term (chronic) effect of the treatment may be enhanced.

According to some embodiments, the combination therapy may include FU treatment performed along with a microwave radiation procedure. Microwave radiation procedure (hereinafter, microwave procedure) may include the external application of microwave radiation at various frequencies (such as for example 6 GHz) to target areas. Target areas may include the fat tissue and/or surrounding tissue and other related tissue, such as connective tissue, muscle tissue, blood vessels, nerve tissue, and the like. For example, U.S. Pat. No. 5,143,063 discloses a method for focusing radiant energy (such as, for example microwave energy) to remove adipose tissue. For example, U.S. Pat. No. 4,597,379 discloses a method of coagulating a muscle tissue contained within fat tissue substantially without harming the fat tissue comprising microwave radiation through the fat tissue towards the muscle tissue at a selective frequency. In accordance, FU treatment may be performed at any time frame relative to the application of the microwave procedure. For example, the FU treatment may be performed at any time frame of about 8 weeks before the FU treatment to about 8 weeks after the FU treatment. The microwave procedure may include power output in the range of, for example 10 to 80 Watt. For example, the microwave procedure may include power output in the range of, for example 30 to 50 Watt. The microwave energy may be directed, for example to muscle tissue that is in contact with the fat tissue that is targeted by the FU treatment. As a result of the microwave procedure, the fat tissue may be more accessible to the FU treatment. The effect of the combination therapy may result in a higher number of fat cell deaths and in a larger volume of fat tissue that has been lysed. In addition, the length of treatment needed to obtain a desirable effect may be shorter in the combination therapy than that needed to obtain a similar effect by each treatment by itself. In addition, the long-term (chronic) effect of the treatment may be enhanced.

According to some embodiments, the combination therapy may include FU treatment performed along with a radio-frequency procedure. Radio frequency (RF) procedure (herein after, RF procedure) may include the external application of RF to target areas. Target areas may include the fat tissue and/or surrounding tissue and other related tissue, such as skin, connective tissue, muscle tissue, blood vessels, nerve tissue, and the like. For example, US patent application No. 2006/0036300 discloses a method of delivering RF energy sufficiently deep below the skin surface so as to generate a heating of the deep skin layer that is strong enough to destroy fat cells. In accordance, FU treatment may be performed at any time frame relative to the application of the RF procedure. For example, the FU treatment may be performed about 3 months before the RF procedure to about 3 months after the RF Procedure. The RF procedure may include one or more sessions of treatment at various intervals, such as for example, three sessions—one month apart. The FU treatment may be performed at any time period relative to each of the sessions of the RF procedure or relative to the first and/or last sessions of the RF procedure. The RF procedure may be performed at a frequency in the range of about 10 to 80 MHz. For example, the RF procedure may be performed at a frequency in the range of about 20 to 60 MHz. RF procedure may be performed at a frequency in the range of about 30 to 50 MHz. RF procedure may be performed at a frequency of about 40.7 MHz. The RF procedure may be performed with power in the range of, for example, 40 to 220 Watts. For example, the RF procedure may be performed with power in the range of, for example, 60 to 200 Watts. For example, the RF procedure may be performed with power in the range of, for example, 90 to 150 Watts. Duration time of each of the RF treatments may include any time at the range of 10 to 30 minutes. For example, the duration may be 20 minutes. Exposure time to the RF energy may be in the range of, for example, about 10 to 40 seconds per designated target area. The RF energy may be directed, for example to the same target area as that targeted by the FU. As a result of the RF procedure, the fat tissue may be more accessible to the FU treatment. The effect of the combination therapy may result in a higher number of fat cell deaths and in a larger volume of fat tissue that has been lysed. In addition, the length of treatment needed to obtain a desirable effect may be shorter in the combination therapy than that needed to obtain a similar effect by each treatment by itself. In addition, the long-term (chronic) effect of the treatment may be enhanced.

According to some embodiments, the combination therapy may include FU treatment performed along with a procedure that involves the use of infra-red (IR) energy (hereinafter IR procedure). The IR procedure may include the external application of IR energy to target areas. The IR applied may include various wavelengths that may range from about 750 nm to 1900 nm. Advantageously, IR at the far infrared energy range (such as 8.0 to 15.0 microns long wave) may be used such that subcutaneous penetration may reach deeper and uniformly. Application of IR energy to target areas may induce, for example, heating of the targeted area and as a result, fat tissue may be more accessible to FU treatment. In accordance, FU treatment may be performed at any time frame relative to the application of the IR procedure. For example, the FU treatment may be performed about 3 months before the IR procedure to about 3 months after the IR Procedure. The IR procedure may include one or more sessions of treatment at various intervals, such as for example, two sessions—one month apart. The FU treatment may be performed at any time period relative to each of the sessions of the IR procedure or relative to the first and/or last sessions of the IR procedure. The IR procedure may include providing energy at the range of about 10 to 80 J/Cm². For example, the energy provided by the IR procedure may by in the range of about 20 to 50 J/Cm². For example, the energy provided by the IR procedure may by in the range of about 30 to 40 J/Cm². The IR procedure may include pulse duration of, for example, 5 to 15 seconds, with about 200 to 500 repeats of the pulses, wherein each pulse may cover a target area of, for example, 1 to 2 c^{m}. The IR energy may be directed, for example to the same target area as that targeted by the FU. The effect of the combination therapy may result in a higher number of fat cell deaths and in a larger volume of fat tissue that has been lysed. In addition, the length of treatment needed to obtain a desirable effect may be shorter in the combination therapy than that needed to obtain a similar effect by each treatment by itself. In addition, the long-term (chronic) effect of the treatment may be enhanced.

According to some embodiments, the combination therapy may include FU treatment performed along with a procedure that includes physical contact/stimulation of a skin region. The physical contact/stimulation procedure may include, for example, any form of skin stimulation, such as massaging, lymphatic massaging, and the like, and may be performed by hand and/or by aid of various devices. The physical contact/stimulation procedure (hereinafter, physical procedure) may be used to reduce fatty tissue. The physical procedure may include dip, drainage massaging procedures, vacuum based massaging procedures, and the like. For example, US patent application No. 2005/0192521 discloses a method of reducing fatty tissue using lymphatic massage. In accordance, FU treatment may be performed at any time period relative to the application of the physical procedure. For example, the FU treatment may be performed at any time range of about 10 days before the physical procedure to about 10 days after the physical procedure. The physical procedure may include, for example, lymphatic massage, and may be directed to a same target area as that targeted by the FU. For example, the massage may include application of force (pressure) in the range of about 40 to 200 mmHg, at a duration of, for example, 10 to 45 minutes per session. The effect of the combination therapy may result in a higher number of fat cell deaths and in a larger volume of fat tissue that has been lysed. In addition, the length of treatment needed to obtain a desirable effect may be shorter in the combination therapy than that needed to obtain a similar effect by each treatment by itself. In addition, the long-term (chronic) effect of the treatment may be enhanced.

According to some embodiments, any combination and any number of the procedures described hereinabove may be performed along with FU treatment. For example, FU treatment may be performed along with administration of an active substance and along with a surgical procedure in one combination therapy. Furthermore, any of the treatments may be repeated any number of times, at any time interval during the combination therapies.

Combination Therapy with SU Treatment

According to some embodiments, there is provided a combination therapy that may include SU treatment and at least one additional therapeutic procedure (treatment) that may be used in combination with the SU treatment. The combination therapy may be used to affect fat cells and tissue by providing a deleterious effect to the fat cells and tissue. The deleterious effect on fat cells and tissue achieved by the combination therapy may be an enhanced effect as compared to the effect achieved by each of the procedures when performed individually. The SU treatment and the at least one additional treatment may be given at the exact same location (region), at locations (regions) that are in close proximity, at regions that are distant (such as different body parts), and any combination thereof.

According to some embodiments, the combination therapy may be performed at any time period in the range of, about 4 months before the SU treatment to about 4 months after the SU treatment. For example, the combination therapy may be performed simultaneously, wherein the SU treatment and the at least one additional therapeutic procedure are performed substantially at the same time, within the range of about 0 seconds to 10 minutes. The combination therapy may be performed such that the SU treatment is performed before the at least one additional therapeutic procedure is performed. The therapeutic procedure may be performed at a time difference in the range of between about 10 minutes to about four months after the SU treatment was performed. The combination therapy may be performed such that the SU treatment is performed after the at least one additional therapeutic procedure was given. The therapeutic procedure may be performed at a time difference in the range of between about 10 minutes to about four months before the SU treatment is performed.

According to some embodiments, the combination therapy may include SU treatment and administration of at least one substance/material. The substance/material that may be administered may be in the form of, for example, but not limited to: fluids, liquids, gels, ointments, creams, pharmaceutical compositions, microparticles and any combination thereof. Administration may include any administration route, such as for example, but not limited to: ingestion, inhalation, injection, subcutaneous injection, anointment, topical administration, spreading and any combination thereof.

According to some embodiments, administration of the substance/material may be performed at the same time as the SU is performed. Administration of the substance material may be performed before or after the SU is performed.

According to some embodiments, the substance that may be administered in combination with SU treatment may include an active substance, such as a drug, toxin, enzyme, biomolecule and the like, and any combination thereof. The active substance may be in the form of a pharmaceutical composition and may include an active substance that may be directed against fat cells, surrounding tissue, such as connective tissue, muscle tissue, nerves, blood vessels and the like, and any combination thereof. For example, the active substance that may be used in combination therapy may include, for example, botulinum toxin type A. Botulinum toxin A is widely used in cosmetics and esthetic medicine, usually in a form known as Botox. One of the effects of Botulinum toxin A is upon muscles, wherein the toxin has the ability to decrease muscle activity by blocking the release of acetylcholine at the neuromuscular junction, thereby rendering the muscle unable to contract for a period of time. In accordance, minute amounts of botulinum toxin A may be administered by injection (hereinafter, botulinum treatment) into the muscle tissue that is located in close proximity to the adipose tissue that may be targeted by the SU. Localized administration of botulinum toxin A (such as in the form of Botox) may include any number of units of Botox, such as in the range of about 1 to 600 units. For example, 20 to 400 units of Botox may be administered. For example, 50 to 300 units of Botox may be administered. For example, 100 to 200 units of Botox may be administered. The Botox may be administered at various time frames in relation to the SU treatment. For example, the Botox may be administered prior to the SU treatment. For example, the Botox may be administered up to six weeks prior to the SU treatment. For example, the Botox may be administered four weeks prior to the SU treatment. For example, the Botox may be administered two weeks prior to the SU treatment. The Botox may be administered thereat with the SU treatment. For example, the Botox may be administered on the same day as the SU treatment, in the range of 4 hours before the SU treatment to 4 hours after the SU treatment. The Botox may be administered at any time frame after the SU treatment. For example, the Botox may be administered up to 6 weeks after the SU treatment. For example, the Botox may be administered four weeks after the SU treatment. For example, the Botox may be administered 2 weeks after the SU treatment. For example, the Botox may be administered 6 days after the SU treatment. The effect of the combination therapy may result in a higher number of fat cell deaths and in a larger volume of fat tissue that has been lysed. In addition, the length of treatment needed to obtain a desirable effect may be shorter in the combination therapy than that needed to obtain a similar effect by each treatment by itself. In addition, the long-term (chronic) effect of the treatment may be enhanced.

According to some embodiments, the active substance that may be used in combination therapy may include, for example, an enzyme, such as Collagenase. Collagenase is a well-known enzyme that may break (proteolyse) the protein collagen, which is the major constituent of connecting tissue. In accordance, Collagenase may be administered by injection (hereinafter, Collagenase treatment) into the connecting tissue that is located in close proximity to the adipose tissue that may be targeted by the SU. Localized administration of Collagenase may include 0.05 to 3.0 mg (milligram) per injection. For example, 0.1 to 2 mg of Collagenase may be administered. For example, 0.3 to 1.5 mg of Collagenase may be administered. For example, 0.5 to 1 mg of Collagenase may be administered. The Collagenase may be administered at any time frame relative to the SU treatment. For example, the Collagenase may be administered up to 14 weeks before the SU treatment. For example, the Collagenase may be administered about 12 weeks before the SU treatment. For example, the Collagenase may be administered about 10 weeks before the SU treatment. For example, the Collagenase may be administered about 8 weeks before the SU treatment. For example, the Collagenase may be administered about 6 weeks before the SU treatment. For example, the Collagenase may be administered about 4 weeks before the SU treatment. For example, the Collagenase may be administered about 2 weeks before the SU treatment. The Collagenase may be administered thereat with the SU treatment. For example, the Collagenase may be administered on the same day as the SU treatment, in the range of about 6 hours before the SU treatment to about 6 hours after the SU treatment. The Collagenase may be administered at any time frame after the SU treatment. For example, the Collagenase may be administered up to about 14 weeks after the SU treatment. For example, the Collagenase may be administered about 12 weeks after the SU treatment. For example, the Collagenase may be administered about 10 weeks after the SU treatment. For example, the Collagenase may be administered about 8 weeks after the SU treatment. For example, the Collagenase may be administered about 6 weeks after the SU treatment. For example, the Collagenase may be administered 4 weeks after the SU treatment. For example, the Collagenase may be administered about 2 weeks after the SU treatment. For example, the Collagenase may be administered about 7 days after the SU treatment. For example, the Collagenase may be administered up to about 6 days after the SU treatment. As a result of the Collagenase treatment, the connective tissue surrounding the adipose tissue are at least partially relaxed, making the adipose tissue more accessible to the SU treatment. In another mode of operation, Collagenase may be administered by injection (hereinafter, Collagenase treatment) into the fat tissue that is targeted by the SU. As a result of the Collagenase treatment, the fat tissue may be more accessible to the SU treatment. The effect of the combination therapy may result in a higher number of fat cell deaths and in a larger volume of fat tissue that has been lysed. In addition, the length of treatment needed to obtain a desirable effect may be shorter in the combination therapy than that needed to obtain a similar effect by each treatment by itself. In addition, the long-term (chronic) effect of the treatment may be enhanced.

According to some embodiments, the active substance that may be used in combination therapy may include, for example, a protein, such as Adiponectin. Adiponectin is a protein that may modulate fat cells and has been suggested as having the ability to decrease fat in adipocytes or the number of adipocytes (such as described in publication No. WO/02072149). In accordance, Adiponectin may be administered by, for example, injection, enteral and/or parenteral routes (hereinafter, Adiponectin treatment) into the adipose tissue that is be targeted by the SU. Adiponectin may be administered at any time period relative to the SU treatment. The effect of the combination therapy may result in a higher number of fat cell deaths and in a larger volume of fat tissue that has been lysed. In addition, the length of treatment needed to obtain a desirable effect may be shorter in the combination therapy than that needed to obtain a similar effect by each treatment by itself. In addition, the long-term (chronic) effect of the treatment may be enhanced.

According to some embodiments, the substance that may be administered in combination therapy with SU may include an inert fluid, such as a saline. Saline may be administered by injection into the fat tissue that is to be targeted by SU. Saline may be injected into the fat tissue at any tonicity range, such as, for example, isotonic, hypertonic and/or hypotonic. Saline injection into the tissue may result is spatial separation of surrounding tissue from fat tissue, making the fat tissue more accessible to SU treatment, by creating a more advantageous surface. Moreover, due to the spatial separation of fat tissue from surrounding tissue, better propagation of the ultrasound energy (waves) may be achieved. As a result, an enhanced effect may be observed. The effect of the combination therapy may result in a higher number of fat cell deaths and in a larger volume of fat tissue that has been lysed. In addition, the length of treatment needed to obtain a desirable effect may be shorter in the combination therapy than that needed to obtain a similar effect by each treatment by itself. In addition, the long-term (chronic) effect of the treatment may be enhanced.

According to some embodiments, the substance that may be administered in combination therapy with SU may include ointments that may be used for the reduction of fat deposits. The ointment may include active substances (ingredients) that may be used for selective reduction of regional fat deposits. The active ingredients may include for example, but not limited to: Caffeine, L-carnitine, Algeae Extracts, Bladderwrack extract, Capsium extract, Green tea extract, Retinol A, Shea Butter, Horsetail Extract, Caprylic triglyceride, Cyclomethicon, Dimethicone, Hydrogenated lecitin, Acrylates C10-30 Alkyl Acrylate crosspolymer, Imidazolidinyl Urea, Triethanolamine, Methylparaben, and the like, and any combinations thereof. For example, the ointment may include commercially available anticellulite creams, such as, for example, Revitol, Sculpt and Tone™, and the like that may include various combinations of active substances that may be used for selective reduction of regional fat deposits. For example, the ointment may further include alpha-2 adrenergic inhibitor (such as described in U.S. Pat. No. 4,588,724). In accordance, the ointment may be administered by, for example, topical spreading (herein after ointment treatment) on the skin region, which resides in correlation to the location of the fat tissue that is to be targeted by the SU treatments. Localized administration of ointment may include topical administration of the ointment, such as by massaging the ointment for 30 to 60 seconds, until at least partially absorbed. The ointment may be administered at any time frame relative to the SU treatment, such as any time in the range of about 60 days before the SU treatment to about 60 days after the SU treatment. The effect of the combination therapy may result in a higher number of fat cell deaths and in a larger volume of fat tissue that has been lysed. In addition, the length of treatment needed to obtain a desirable effect may be shorter in the combination therapy than that needed to obtain a similar effect by each treatment by itself. In addition, the long-term (chronic) effect of the treatment may be enhanced.

According to some embodiments, the combination therapy may include SU treatment performed along with cryosurgery procedure. Cryosurgery is a procedure for destroying tissue, by freezing the tissue to be destroyed. This is a minimally invasive technique, wherein a cryogen cooled probe may be inserted into a desired tissue, consequently freezing the tissue. For example, U.S. Pat. No. 6,032,675 discloses a method for removing fatty tissue in a body, combining cryosurgery and liposuction. In accordance, SU treatment may be performed at any time period relative to the cryosurgery procedure. In this mode, fat cells and tissue are damaged by the SU treatment and the cryosurgery procedure. Thus, the effect of the combination therapy may result in a higher number of fat cell deaths and in larger volume of fat tissue that has been lysed. In addition, the length of treatment needed to obtain a desirable effect may be shorter in the combination therapy than that needed to obtain a similar effect by each treatment by itself. In addition, the long-term (chronic) effect of the treatment may be enhanced.

According to some embodiments, the combination therapy may include SU treatment in addition to at least one non-invasive procedure. The non-invasive procedure may include, for example, energy based procedures such as laser based procedure, infra-red based procedure, microwave-energy based procedures, radio frequency based procedures, and the like. The non-invasive procedure may include, for example, physical contact, direct contact and the like, that may be applied to an outer surface, such as skin, that correspond to the subcutaneous fat tissue that may be targeted by the SU treatment.

According to some embodiments, the combination therapy may include SU treatment performed along with a laser-based procedure. Laser based procedure may include, for example, the external application of laser energy thorough the skin to target areas in the body, such as fat tissue. For example, US patent application No. 2005/0203594 discloses a non invasive method of reducing fat from targeted regions of a patient's body by applying low-level laser energy externally through the skin of the patient to the targeted areas. In accordance, SU treatment may be performed at any time frame relative to the application of the laser energy (hereinafter, laser procedure). For example, the laser procedure may be performed in the range of about 8 weeks before the SU treatment to about 8 weeks after the SU treatment. The laser procedure may be performed at various wavelengths, such as for example, in the range of about 515 to 1200 nm. For example, the laser procedure may be performed with a wavelength in the range of about 550 to 900 nm. For example, the laser procedure may be performed with a wavelength in the range of about 600 to 800 nm. For example, the laser procedure may be performed with a wavelength of about 635 nm. The laser procedure may be performed with a pulse duration range of about 0.2 to 100 milliseconds. For example, the laser procedure may be performed with a pulse duration range of about 0.5 to 88.5 milliseconds. For example, the laser procedure may be performed with a pulse duration range of about 1 to 75 milliseconds. For example, the laser procedure may be performed with a pulse duration range of about 20 to 50 milliseconds. For example, the laser procedure may be performed with a pulse duration range of about 30 to 40 milliseconds. The laser procedure may be performed with a pulse duration range of about 1 to 100 mJ/cm². For example, the laser procedure may be performed with a pulse duration range of about 3 to 70 mJ/cm². For example, the laser procedure may be performed with a pulse duration range of about 20 to 50 mJ/cm². For example, the laser procedure may be performed with a pulse duration range of about 30 to 40 mJ/cm². The SU treatment and the laser procedure may be directed to the same or different target areas. The effect of the combination therapy may result in a higher number of fat cell deaths and in a larger volume of fat tissue that has been lysed. In addition, the length of treatment needed to obtain a desirable effect may be shorter in the combination therapy than that needed to obtain a similar effect by each treatment by itself. In addition, the long-term (chronic) effect of the treatment may be enhanced.

According to some embodiments, the combination therapy may include SU treatment performed along with microwave radiation procedure. Microwave radiation procedure (hereinafter, microwave procedure) may include the external application of microwave radiation at various frequencies (such as, for example, 6 GHz) to target areas. Target areas may include the fat tissue and/or surrounding tissue and other related tissue, such as connective tissue, muscle tissue, blood vessels, nerve tissue, and the like. For example, U.S. Pat. No. 5,143,063 discloses a method for focusing radiant energy (such as, for example, microwave energy) to remove adipose tissue. For example, U.S. Pat. No. 4,597,379 discloses a method of coagulating a muscle tissue contained within fat tissue substantially without harming the fat tissue comprising microwave radiation through the fat tissue towards the muscle tissue at a selective frequency. In accordance, SU treatment may be performed at any time frame relative to the application of the microwave procedure. For example, the SU treatment may be performed at any time frame of about 8 weeks before the SU treatment to about 8 weeks after the SU treatment. The microwave procedure may include power output in the range of, for example 10 to 80 Watt. For example, the microwave procedure may include power output in the range of, for example 30 to 50 Watt. The microwave energy may be directed, for example to muscle tissue that is in contact with the fat tissue that is targeted by the SU treatment. As a result of the microwave procedure, the fat tissue may be more accessible to the SU treatment. The effect of the combination therapy may result in a higher number of fat cell deaths and in a larger volume of fat tissue that has been lysed. In addition, the length of treatment needed to obtain a desirable effect may be shorter in the combination therapy than that needed to obtain a similar effect by each treatment by itself. In addition, the long-term (chronic) effect of the treatment may be enhanced.

According to some embodiments, the combination therapy may include SU treatment performed along with radio-frequency procedure. Radio frequency (RF) procedure (herein after, RF procedure) may include the external application of RF to target areas. Target areas may include the fat tissue and/or surrounding tissue and other related tissue, such as skin, connective tissue, muscle tissue, blood vessels, nerve tissue, and the like. For example, US patent application No. 2006/0036300 discloses a method of delivering RF energy sufficiently deep below the skin surface so as to generate a heating of the deep skin layer that is strong enough to destroy fat cells. In accordance, SU treatment may be performed at any time frame relative to the application of the RF procedure. For example, the SU treatment may be performed about 3 months before the RF procedure to about 3 months after the RF procedure. The RF procedure may include one or more sessions of treatment at various intervals, such as for example, three sessions—one month apart. The SU treatment may be performed at any time period relative to each of the sessions of the RF procedure or relative to the first and/or last sessions of the RF procedure. The RF procedure may be performed at a frequency in the range of about 10 to 80 MHz. For example, the RF procedure may be performed at a frequency in the range of about 20 to 60 MHz. RF procedure may be performed at a frequency in the range of about 30 to 50 MHz. RF procedure may be performed at a frequency of about 40.7 MHz. The RF procedure may be performed with power in the range of, for example, 40 to 220 Watts. For example, the RF procedure may be performed with power in the range of, for example, 60 to 200 Watts. For example, the RF procedure may be performed with power in the range of, for example, 90 to 150 Watts. Duration time of each of the RF treatments may include any time period at the range of 10 to 30 minutes. For example, the duration may be 20 minutes. Exposure time to the RF energy may be in the range of, for example, about 10 to 40 seconds per designated target area. The RF energy may be directed, for example to the same target area as that targeted by the SU. As a result of the RF procedure, the fat tissue may be more accessible to the SU treatment. The effect of the combination therapy may result in a higher number of fat cell deaths and in a larger volume of fat tissue that has been lysed. In addition, the length of treatment needed to obtain a desirable effect may be shorter in the combination therapy than that needed to obtain a similar effect by each treatment by itself. In addition, the long-term (chronic) effect of the treatment may be enhanced.

According to some embodiments, the combination therapy may include SU treatment performed along with a procedure that involves the use of infrared (IR) energy (hereinafter IR procedure). The IR procedure may include the external application of IR energy to target areas. The IR applied may include various wavelengths that may range from about 750 nm to 1900 nm. Advantageously, IR at the far infrared energy range (such as 8.0 to 15.0 microns long wave) may be used such that subcutaneous penetration may reach deeper and uniformly. Application of IR energy to target areas may induce, for example, heating of the targeted area and as a result, fat tissue may be more accessible to SU treatment. In accordance, SU treatment may be performed at any time frame relative to the application of the IR procedure. For example, the SU treatment may be performed about 3 months before the IR procedure to about 3 months after the IR Procedure. The IR procedure may include one or more sessions of treatment at various intervals, such as for example, two sessions—one month apart. The SU treatment may be performed at any time period relative to each of the sessions of the IR procedure or relative to the first and/or last sessions of the IR procedure. The IR procedure may include providing energy at the range of, about, 10 to 80 J/Cm². For example, the energy provided by the IR procedure may by in the range of, about 20 to 50 J/Cm². For example, the energy provided by the IR procedure may by in the range of, about 30 to 40 J/Cm². The IR procedure may include pulse duration of, for example, 5 to 15 seconds, with about 200 to 500 repeats of the pulses, wherein each pulse may cover a target area of, for example, 1 to 2 c^{m}. The IR energy may be directed, for example to the same target area as that targeted by the SU. The effect of the combination therapy may result in a higher number of fat cell deaths and in a larger volume of fat tissue that has been lysed. In addition, the length of treatment needed to obtain a desirable effect may be shorter in the combination therapy than that needed to obtain a similar effect by each treatment by itself. In addition, the long-term (chronic) effect of the treatment may be enhanced.

According to some embodiments, the combination therapy may include SU treatment performed along with a procedure that includes physical contact/stimulation of a skin region. The physical contact/stimulation procedure may include, for example, any form of skin stimulation, such as massaging, lymphatic massaging, and the like and may be performed by hand and/or by aid of various devices. The physical contact/stimulation procedure (hereinafter, physical procedure) may be used to reduce fatty tissue. The physical procedure may include dip, drainage massaging procedures, vacuum based massaging procedures, and the like. For example, US patent application No. 2005/0192521 discloses a method of reducing fatty tissue using lymphatic massage. In accordance, SU treatment may be performed at any time period relative to the application of the physical procedure. For example, the SU treatment may be performed at any time range of about 10 days before the physical procedure to about 10 days after the physical procedure. The physical procedure may include, for example, lymphatic massage, and may be directed to a same target area as that targeted by the SU. For example, the massage may include application of force (pressure) in the range of about 40 to 200 mmHg, at a duration of, for example, 10 to 45 minutes per session. The effect of the combination therapy may result in a higher number of fat cell deaths and in a larger volume of fat tissue that has been lysed. In addition, the length of treatment needed to obtain a desirable effect may be shorter in the combination therapy than that needed to obtain a similar effect by each treatment by itself. In addition, the long-term (chronic) effect of the treatment may be enhanced.

According to some embodiments, any combination and any number of the procedures described above herein may be performed along with SU treatment. For example, SU treatment may be performed along with administration of an active substance and along with a surgical procedure in one combination therapy. Furthermore, any of the treatments may be repeated any number of times, at any time interval during the combination therapies.

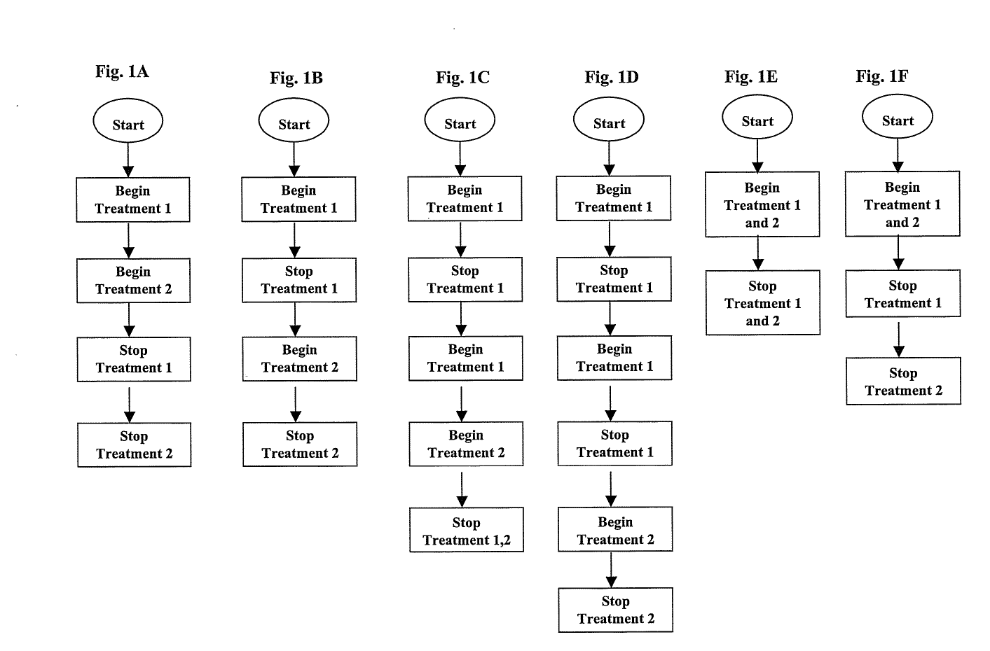

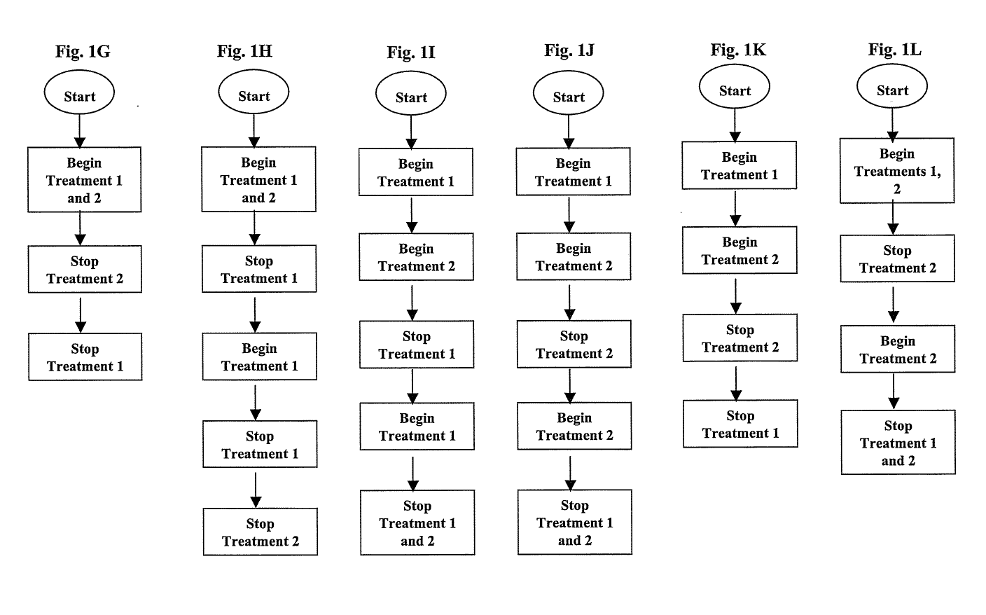

Reference is made to FIG. 1, which illustrates exemplary flow diagrams of a method of combination therapy, according to some embodiments. Various combinations of therapies may be performed, wherein at least one of the treatments performed include the application of ultrasonic energy, such as focused ultrasonic energy and/or surface ultrasonic energy. For example, two treatments may be combined, such as shown by way of example in FIGS. 1A-L. The treatments may include any treatment at any time interval between the treatments, such as in the range of 0 seconds to 4 months, depending on the chosen combination of treatments. For example, as shown in FIG. 1A, the first treatment is commenced. After a time period of 10 minutes to four months the second treatment is commenced. Then, the first treatment may be stopped, after which the second treatment is stopped. For example, the first treatment may include administration of a substance and the second treatment may include application of ultrasonic energy. As shown in FIG. 1B, the first treatment is commenced. After a predetermine time period (such as in the range of 10 minutes to four months) the first treatment is stopped. Thereafter, after a delay of 10 minutes to four hours, the second treatment may commence, and then, after a predetermined time (such as in the range of 10 minutes to four months), the second treatment is stopped. For example, the first treatment may include an invasive procedure, such as, for example, liposuction and the second treatment may include application of ultrasonic energy. As shown in FIG. 1C, the first treatment is commenced. After a predetermine time period (such as in the range of 10 minutes to four months) the first treatment is stopped. Thereafter, after a predetermined time period (such as in the range of 10 minutes to four months) the first treatment is commenced again. Thereafter, after a predetermined time period (such as in the range of 10 minutes to 4 months), the second treatment may commence. Then, both the first and the second treatment may be stopped. For example, the first treatment may include non-invasive treatment, such as IR treatment and the second treatment may include application of ultrasonic energy. As shown in FIG. 1D, the first treatment is commenced. After a predetermine time period (such as in the range of 10 minutes to four months) the first treatment is stopped. Thereafter, after a predetermined time period (such as in the range of 10 minutes to four months) the first treatment is commenced again. After a predetermine time period (such as in the range of 10 minutes to four months) the first treatment is stopped. Thereafter, the second treatment may commence and after a predetermined time period may be stopped. For example, the first treatment may include non-invasive treatment, such as RF treatment and the second treatment may include application of ultrasonic energy. As shown in FIG. 1E, the first and second treatments are commenced simultaneously (such as in the range of 0 seconds to 10 minutes). Then, after a predetermined time period the treatments are simultaneously (in the range of 0 seconds to 10 minutes) stopped. For example, the first treatment may include application of ultrasonic energy and the second treatment may include application of a substance, such as an anti cellulite cream ointment. As shown in FIG. 1F, the first and second treatments are commenced simultaneously (such as in the range of 0 seconds to 10 minutes). Then, after a predetermined time period the first treatment is stopped. Then, after a time period in the range of 10 minutes to four months, the second treatment is stopped. For example, the first treatment may include application of ultrasonic energy and the second treatment may include non-invasive treatment, such as laser based treatment. As shown in FIG. 1G, the first and second treatments are commenced simultaneously (such as in the range of 0 seconds to 10 minutes). Then, after a time period in the range of 10 minutes to four months, the first treatment is stopped. As shown in FIG. 1H, the first and second treatments are commenced simultaneously (such as in the range of 0 seconds to 10 minutes). Then, after a predetermined time period the first treatment is stopped. Then, after a time period in the range of 10 minutes to four months, the first treatment is commenced again. Then, after a time period in the range of 10 minutes to four months, the first treatment is stopped. After a time period of 10 minutes to four months, the second treatment is stopped. For example, the first treatment may include application of ultrasonic energy and the second treatment may include administration of substance, such as, for example, Botox. As shown in FIG. 1I, the first treatment is commenced. After a time period of 10 minutes to four months the second treatment is commenced. After a predetermined time period, the first treatment is stopped. After an additional predetermined time period the first treatment is commenced again. After a predetermined time period the first and second treatments are simultaneously (such as in the range of 0 seconds to 10 minutes) stopped. For example, the first treatment may include application of ultrasonic energy and the second treatment may include non-invasive treatment, such as physical stimulation of the skin, such as for example, drainage massaging. As shown in FIG. 1J, the first treatment is commenced. After a time period of 10 minutes to four months the second treatment is commenced. After a predetermined time period the second treatment is stopped. After additional predetermined time period the second treatment is commenced again. After a predetermined time period the first and second treatments are simultaneously (such as in the range of 0 seconds to 10 minutes) stopped. For example, the first treatment may include application of ultrasonic energy and the second treatment may include non-invasive treatment, such as application of microwave energy. As shown in FIG. 1K, the first treatment is commenced. After a time period of 10 minutes to four months the second treatment is commenced. Thereafter, after a predetermined time period the second treatment is stopped. After a time period of 10 minutes to four months the first treatment is stopped. For example, the first treatment may include administration of a substance, such as Saline and the second treatment may include application of ultrasonic energy. As shown in FIG. 1L, the first and second treatments are commenced simultaneously (such as in the range of 0 seconds to 10 minutes). Then, after a predetermined time period the second treatment is stopped. Then, after a time period in the range of 10 minutes to four months, the second treatment is commenced again. Then, after a time period in the range of 10 minutes to four months, the first and second treatments are stopped simultaneously (in the range of 0 seconds to 10 minutes). For example, the first treatment may include application of ultrasonic energy and the second treatment may include no-invasive treatment, such as application of physical stimulation, such as, for example, lymphatic massaging.

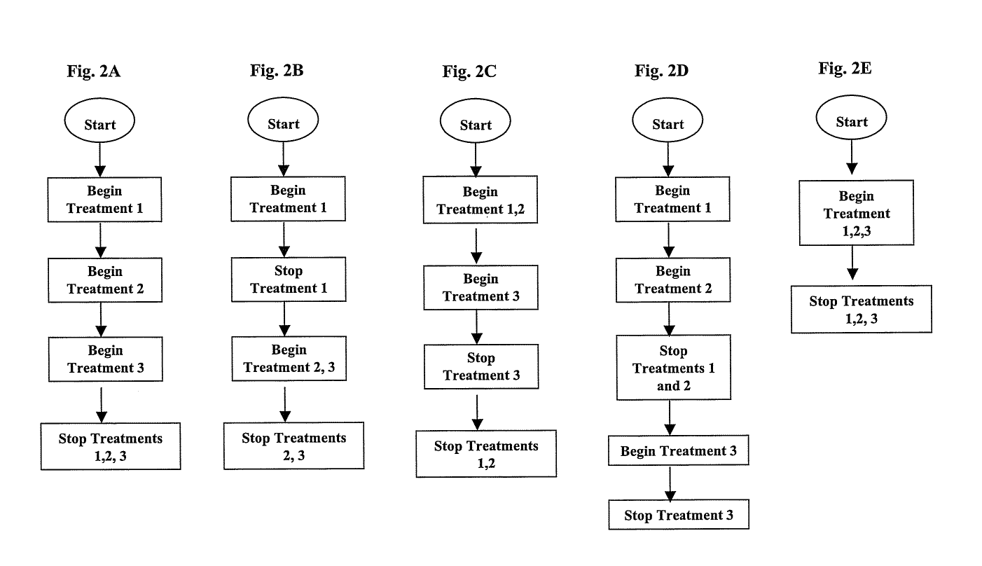

According to some embodiments, more than one treatment may be combined with application of ultrasonic energy. Reference is now made to FIG. 2, which illustrates exemplary flow diagrams of a method of combination therapy, according to some embodiments. Various combinations of therapies may be performed, wherein at least one of the treatments performed include the application of ultrasonic energy, such as focused ultrasonic energy and/or surface ultrasonic energy. For example, three treatments may be combined, such as shown by way of example in FIGS. 2A-E. The treatments may include any treatment at any time interval between the treatments, such as in the range of 0 seconds to 4 months, depending on the chosen combination of treatments. For example, as shown in FIG. 2A, the first treatment is commenced. Then, after a predetermined time period (such as in the range of 10 minutes to 4 months), the second treatment is commenced. Then, after a predetermined time period, the third treatment is commenced, after which time of 10 minutes to 4 months, the treatments are stopped simultaneously (such as in the range of 0 seconds to 10 minutes). For example, in such a setting the first treatment may include administration of a substance, such as Botox. The second treatment may include application of ultrasonic energy. The third treatment may include application of non-invasive treatments, such as, for example, lymphatic massaging. For example, as shown in FIG. 2B, the first treatment is commenced. After a predetermined treatment time, the treatment is stopped. Then, after a time period (such as in the range of 10 minutes to 4 months), the second and third treatments are commenced simultaneously (such as in the range of 0 seconds to 10 minutes). After a predetermined treatment time, treatments 2 and 3 are stopped simultaneously (such in the range of 0 seconds to 10 minutes). For example, the first treatment may include an invasive surgical procedure, such as, for example, liposuction. The second treatment may include application of a non-invasive treatment, such as microwave energy. The third treatment may include application of ultrasonic energy. For example, as shown in FIG. 2C, the first and second treatments are commenced simultaneously (such as in the range of 0 seconds to 10 minutes). Then, after a time period in the range of 10 minutes to four months, the third treatment is commenced. After a predetermined time period the third treatment is stopped. Then, after a time period, the first and second treatments are stopped. For example, the first treatment may include application of a substance, such as, for example, Collagenase treatment. The second treatment may include administration of a substance, such as, for example, saline. The third treatment may include application of ultrasonic energy. For example, as shown in FIG. 2D, the first is commenced. Then, after a time period of 10 minutes to four months, the second treatment is commenced. Thereafter, after a predetermined time period, the first and second treatments are stopped. Thereafter, after a time period of about 10 minutes to four months, the third treatment is commenced and stopped after a predetermined time period. For example, the first treatment may include application of a substance, such as, for example, saline. The second treatment may include application of an invasive treatment, such as, for example, cryosurgery. The third treatment may include application of ultrasonic energy. For example, as shown in FIG. 2E, the first, second and third treatments are commenced simultaneously (such as in the range of 0 seconds to 10 minutes). Then, after a time period in the range of 10 minutes to four months, all three treatments are simultaneously stopped (with a time difference of 0 seconds to 10 minutes between the stopping of the treatments). For example, the first treatment may include application of non-invasive treatments, such as use of an anti-cellulite ointment. The second treatment may include application of ultrasonic energy. The third treatment may include application of a non-invasive treatment, such as, for example, application of RF treatment.

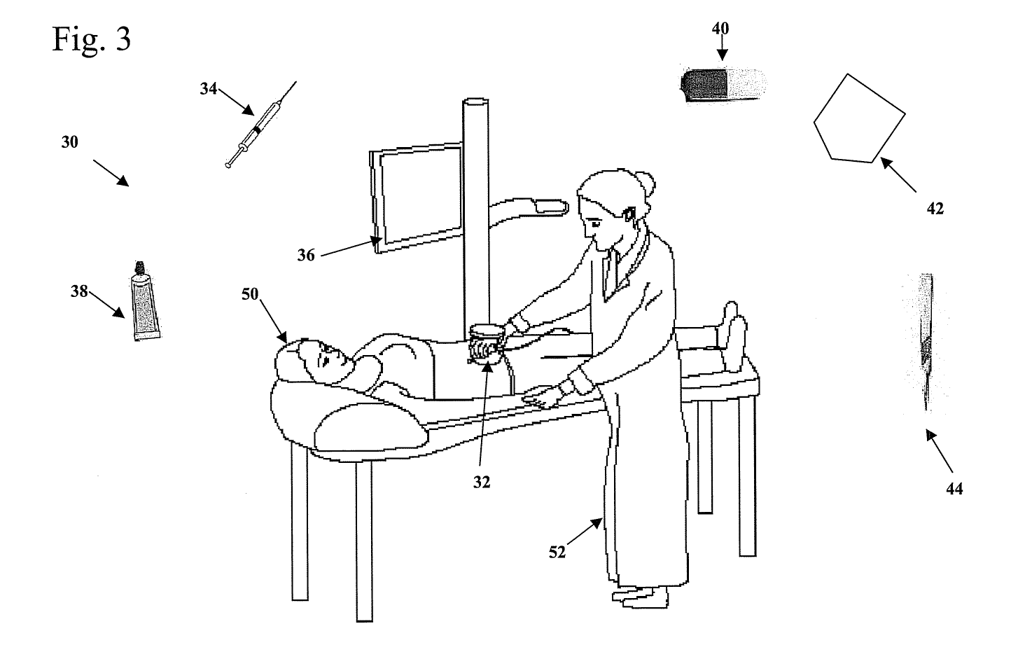

According to some embodiments, there is provided a system for combination therapy for producing lysis of adipose tissue that includes a transducer adapted to apply ultrasonic energy to said adipose tissue and at least one additional unit adapted to provide a therapeutic procedure to said adipose tissue or surrounding tissue(s). The ultrasonic energy may include focused ultrasonic energy and/or surface ultrasonic energy. Reference is now made to FIG. 3, which illustrates a system for combination therapy, according to some embodiments. As shown in FIG. 3, system 30 may include an ultrasonic transducer, such as transducer 32. System 30 may further include at least one additional unit that may be adapted to provide additional treatment (procedure). The at least one unit may include any type of dispenser, surgical apparatus, pressure providing apparatus (such as vacuum apparatus), non-invasive apparatus, and the like. For example, a unit, such as unit 34 may be adapted to administer, by injection, various substances, such as, for example, any of the substances described above herein. For example, a unit, such as unit 38 may be adapted to provide/dispense various substances, such as, for example any of the substances described above herein. For example, a unit, such as unit 40 may be adapted to provide/dispense various medications, drugs, and the like, such as, for example any of the substances described above herein. For example, a unit, such as unit 42 may be adapted to provide various non-invasive treatments, such as, for example, IR treatment, RF treatment, Laser treatment, and the like and any other non-invasive treatment described above herein. For example, a unit, such as unit 44 may be adapted to provide various invasive treatments, such as liposuction, cryosurgery and any of the invasive treatments described above herein. The system, such as system 30 may further include one or more displays, such as display 36, that may be attached to the ultrasonic transducer and/or the at least one additional unit. The ultrasonic transducer may be permanently attached with the at least one additional unit adapted to provide a therapeutic procedure. The ultrasonic transducer may be integrally formed with the at least one additional unit adapted to a provide therapeutic procedure. The ultrasonic transducer may be reversibly attached to at least one additional unit adapted to provide a therapeutic procedure. According to other embodiments, the ultrasonic transducer, such as transducer 30, may not be physically attached to the at least one additional unit adapted to provide a therapeutic procedure. Further shown in FIG. 3, a patient, such as patient 50, who is receiving a combination therapy to produce lysis of adipose tissue and a therapy provider, such as therapy provider 52.

While a number of exemplary aspects and embodiments have been discussed above, those of skill in the art will recognize certain modifications, permutations, additions and sub-combinations thereof. It is therefore intended that the following appended claims and claims hereafter introduced be interpreted to include all such modifications, permutations, additions and sub-combinations as are within their true spirit and scope.

1. A method of combination therapy for producing lysis of adipose tissue comprising:
applying focused ultrasonic energy to said adipose tissue; and
applying at least one additional therapeutic procedure to said adipose tissue or surrounding tissue(s).

2. The method of claim 1, wherein said at least one additional therapeutic procedure comprises administration of a substance, invasive surgical procedure, non-invasive procedure and any combination thereof.

3. The method of claim 2, wherein said substance comprises a drug, toxin, enzyme, biomolecule, inert fluid and any combination thereof.

4. The method of claim 2, wherein said invasive surgical procedure comprises liposuction, ultrasound assisted liposuction, external ultrasonic assisted liposuction, cryosurgery and any combination thereof.

5. The method of claim 2, wherein said non-invasive procedure comprises: laser based procedures, radio frequency based procedures, infra red based procedures, microwave based procedures, massaging based procedures and any combination thereof.

6. The method of claim 1, wherein the focused ultrasonic energy is applied before the at least one additional therapeutic procedure is applied.

7. The method of claim 1, wherein the focused ultrasonic energy is applied at the same time as the at least one additional therapeutic procedure is applied.

8. The method of claim 1, wherein the focused ultrasonic energy is applied after the at least one additional therapeutic procedure is applied.

9. The method of claim 1, further comprising an enhanced effect on said adipose tissue.

10. The method of claim 9 wherein said enhanced effect is increasing the adipose tissue lysis.

11. The method of claim 9, wherein enhanced effect is decreasing treatment time needed to produce adipose tissue lysis.

12. The method of claim 9, wherein said enhanced effect is a synergistic effect.

13. The method of claim 1, wherein said surrounding tissue comprises:
muscle tissue, connective tissue, blood vessels, nerve tissue, adipose tissue and any combination thereof.

14. A method of combination therapy for producing lysis of adipose tissue comprising:
applying surface ultrasonic energy to said adipose tissue; and
applying at least one additional therapeutic procedure to said adipose tissue or surrounding tissue(s).

15. The method of claim 14, wherein said at least one additional therapeutic procedure comprises administration of substance, invasive surgical procedure, non-invasive procedure and any combination thereof.

16. The method of claim 15, wherein said substance comprises a drug, toxin, enzyme, biomolecule, inert fluid and any combination thereof.

17. The method of claim 15, wherein said non-invasive procedure comprises: laser-based procedures, radio frequency based procedures, infra red based procedures, microwave based procedures, massaging based procedures and any combination thereof.

18. The method of claim 14, wherein the surface ultrasonic energy is applied before the at least one additional therapeutic procedure is applied.

19. The method of claim 14, wherein the surface ultrasonic energy is applied at the same time as the at least one additional therapeutic procedure is applied.

20. The method of claim 14, wherein the surface ultrasonic energy is applied after the at least one additional therapeutic procedure is applied.

21. The method of claim 14, further comprising an enhanced effect on said adipose tissue.

22. The method of claim 21 wherein said enhanced effect is increasing the adipose tissue lysis.

23. The method of claim 21, wherein said enhanced effect is decreasing treatment time needed to produce adipose tissue lysis.

24. The method of claim 21, wherein said enhanced effect is a synergistic effect.

25. The method of claim 21, wherein said enhanced effect is an additive effect.

26. The method of claim 16, wherein said surrounding tissue comprises: muscle tissue, connective tissue, blood vessels, nerve tissue, adipose tissue and any combination thereof.

27. The system of claim 53, wherein said ultrasonic energy comprises focused ultrasonic energy.

28. The system of claim 27, wherein said therapeutic procedure comprises administration of a substance, invasive surgical procedure, non-invasive procedure and any combination thereof.

29. The system of claim 28, wherein said substance comprises a drug, toxin, enzyme, biomolecule, inert fluid and any combination thereof.

30. The system of claim 28, wherein said invasive surgical procedure comprises liposuction, ultrasound assisted liposuction, external ultrasonic assisted liposuction, cryosurgery and any combination thereof.

31. The system of claim 28, wherein said non-invasive procedure comprises: laser based procedures, radio frequency based procedures, infra red based procedures, microwave based procedures, massaging based procedures and any combination thereof.

32. The system of claim 27, wherein said focused ultrasonic energy is applied before the at least one additional therapeutic procedure is applied.

33. The system of claim 27, wherein the focused ultrasonic energy is applied at the same time as the at least one additional therapeutic procedure is applied.

34. The system of claim 27, wherein the focused ultrasonic energy is applied after the at least one additional therapeutic procedure is applied.

35. The system of claim 27, further comprising an enhanced effect on said adipose tissue.

36. The system of claim 35 wherein said enhanced effect is increasing the adipose tissue lysis.

37. The system of claim 35, wherein said enhanced effect is decreasing treatment time needed to produce adipose tissue lysis.

38. The system of claim 35, wherein said enhanced effect is a synergistic effect.

39. The system of claim 27, wherein said surrounding tissue comprises: muscle tissue, connective tissue, blood vessels, nerve tissue, adipose tissue and any combination thereof.

40. The system of claim 53, wherein said ultrasonic energy comprises.

41. The system of claim 40, wherein said therapeutic procedure comprises administration of a substance, invasive surgical procedure, non-invasive procedure and any combination thereof.

42. The system of claim 41, wherein said substance comprises a drug, toxin, enzyme, biomolecule, inert fluid and any combination thereof.

43. The system of claim 41, wherein said invasive surgical procedure comprises liposuction, ultrasound assisted liposuction, external ultrasonic assisted liposuction, cryosurgery and any combination thereof.

44. The system of claim 41, wherein said non invasive procedure comprises: laser based procedures, radio frequency based procedures, Infra Red based procedures, microwave based procedures, massaging based procedures and any combination thereof.

45. The system of claim 40, wherein said surface ultrasonic energy is applied before the at least one additional therapeutic procedure is applied.

46. The system of claim 40, wherein the surface ultrasonic energy is applied at the same time as the at least one additional therapeutic procedure is applied.

47. The system of claim 40, wherein the surface ultrasonic energy is applied after the at least one additional therapeutic procedure is applied.

48. The system of claim 40, further comprising an enhanced effect on said adipose tissue.

49. The system of claim 48 wherein said enhanced effect is increasing the adipose tissue lysis.

50. The system of claim 48, wherein said enhanced effect is decreasing treatment time needed to produce adipose tissue lysis.

51. The system of claim 48, wherein said enhanced effect is a synergistic effect.

52. The system of claim 40, wherein said surrounding tissue comprises: muscle tissue, connective tissue, blood vessels, nerve tissue, adipose tissue and any combination thereof.

53. A system for combination therapy for producing lysis of adipose tissue, the system comprising:
a transducer adapted to apply ultrasonic energy to said adipose tissue; and
at least one additional unit adapted to provide a therapeutic procedure to said adipose tissue or surrounding tissue(s).
